(19) United States
(12) Patent Application Publication
(10) Pub. No.: US 2024/0050169 A1
(43) Pub. Date: Feb. 15, 2024

(54) INPUT DEVICE AND SURGICAL ROBOT

(71) Applicant: CORNERSTONE TECHNOLOGY (SHENZHEN) LIMITED, Shenzhen (CN)

(72) Inventors: Rongjian SUN, Shenzhen (CN); Yueqiang LU, Shenzhen (CN); Tian XIE, Shenzhen (CN)

(21) Appl. No.: 18/495,672

(22) Filed: Oct. 26, 2023

Related U.S. Application Data
(63) Continuation of application No. PCT/CN2022/078979, filed on Mar. 3, 2022.

(30) Foreign Application Priority Data
Apr. 28, 2021 (CN) 202110469703.9

(51) Int. Cl. A61B 34/00; A61B 34/37

(52) U.S. Cl. CPC A61B 34/25; A61B 34/37; A61B 2560/02; A61B 2017/00876

(57) ABSTRACT

Disclosed are an input device and a surgical robot. The input device includes a mounting base, a rotation support, a grip mechanism, a connection member, a magnetic element and at least one first detection component. The rotation support is rotatable around a first central axis relative to the mounting base. The grip mechanism is disposed on the rotation support, and is transitionable between an open position and a close position. The connection member is connected with the grip mechanism, and is movable in a direction substantially parallel to the first central axis with opening and closing of the grip mechanism. The magnetic element is disposed on the rotation support or the connection member. The at least one first detection component is disposed on the mounting base, and is configured to detect a magnetic field direction of the magnetic element.

### CROSS REFERENCE TO RELATED APPLICATIONS

This application is a continuation of PCT Patent Application No. PCT/CN2022/078979, entitled “INPUT DEVICE AND SURGICAL ROBOT,” filed Mar. 3, 2022, which claims priority to Chinese Patent Application No. 202110469703.9, entitled “INPUT DEVICE AND SURGICAL ROBOT,” filed on Apr. 28, 2021, each of which is incorporated by reference herein in its entirety.

### TECHNIC FIELD

The various embodiments described in this document relate to the technical field of medical instruments, and in particular to an input device and a surgical robot.

### BACKGROUND

Most medical surgical robots adopt a master-slave control architecture, where a doctor operates a master manipulator of an input device to control a movement of a slave manipulator such as an end effector through remote communication and a computer. For example, the doctor controls the end effector, such as a pair of forceps, a pair of scissors, a clamp, etc., by opening and closing a grip handle of the input device.

For a measurement of a rotation angle of a rotating unit of a known input device, the rotation of the rotating unit is usually transmitted to a bevel gear, and then an angle of the bevel gear is measured by an encoder. Due to a gap between the bevel gears, there is a certain difference between the angle measured by the encoder and an actual rotation angle of the rotating unit, and the angle measured may be inaccurate.

In addition, an included angle between members of a grip mechanism of the input device is an important input parameter for control, so it is extremely important for accuracy and stability of the measurement of the included angle. In the known angle detection method of the grip mechanism, a Hall sensing chip is usually arranged in a certain part of the input device, and a magnetic element is arranged in another part of the input device (for example, a rotation support is provided with the Hall sensing chip, and the grip mechanism is provided with the magnetic element), and the part with the magnetic element moves to change the magnetic field intensity around the Hall sensing chip. However, the change in magnetic field intensity is not linear due to a non-linear variation of a distance between the magnetic element and the Hall sensing chip, thus the included angle cannot be detected directly, and additional sensors are required for angle calibration.

### SUMMARY

A series of simplified concepts have been introduced in the summary section, which will be further elaborated in the embodiments. The summary of this application does not mean attempting to limit the key features and essential technical features of the claimed technical solution, nor does it mean attempting to determine the protection scope of the claimed technical solution.

According to various embodiments of the present disclosure, an input device is provided, including a mounting base, a rotation support, a grip mechanism, a connection member, a magnetic element, and at least one first detection component. The rotation support is rotatable relative to the mounting base around a first central axis. The grip mechanism is disposed on the rotation support and is transitionable between an open position and a close position. The connection member is connected with the grip mechanism and movable in a direction substantially parallel to the first central axis with the opening and closing of the grip mechanism. The magnetic element is disposed on the rotation support or the connection member. The at least one first detection component is disposed on the mounting base, and is configured to detect a magnetic field direction of the magnetic element.

According to the input device provided by the embodiments of the present disclosure, the magnetic element is disposed on the rotation support or the connection member, and the connection member is indirectly connected to the rotation support through the grip mechanism, so the magnetic element can rotate with the rotation of the rotation support to change the position relationship between the first detection component and the magnetic element, resulting in that the magnetic field of the magnetic element is deflected with respect to the first detection component. The first detection component can detect changes in the magnetic field direction and obtain a rotation angle of the rotation support through data calculation, thus the rotation angle of the rotation support can be measured without using other sensors for angle calibration.

In an embodiment, the grip mechanism includes a first grip member and a second grip member which are respectively pivotally disposed on the rotation support.

In an embodiment, the first grip member and the second grip member are disposed on two opposite sides of the rotation support respectively, and the first grip member and the second grip member are cooperatively movable, and/or the connection member has one end to which the first grip member and the second grip member are pivotally connected, and one other end on which the magnetic element is disposed and which is located away from the first grip member and the second grip member.

In an embodiment, the input device further includes a first pin and a second pin substantially parallel to the first pin, the first grip member has one end pivotally connected to the rotation support through the first pin, and the second grip member has one end pivotally connected to the rotation support through the second pin.

In an embodiment, the input device further includes a first connecting rod and a second connecting rod, the first connecting rod has two ends pivotally connected to the first grip member and the connection member, respectively, and the second connecting rod has two ends pivotally connected to the second grip member and the connection member, respectively.

In an embodiment, the input device further includes a third pin, a fourth pin and a fifth pin which are substantially parallel to each other, one of the two ends of the first connecting rod is pivotally connected to the first grip member through the third pin, one of the two ends of the second connecting rod is pivotally connected to the second grip member through the fourth pin, and the other one of the two ends of the first connecting rod and the other one of the two ends of the second connecting rod are pivotally connected to the connection member through the fifth pin.

In an embodiment, the third pin is substantially parallel to the first pin, and/or a central axis of the third pin is substantially perpendicular to the first central axis.

In an embodiment, each of the at least one first detection component is spaced apart from the magnetic element in a direction substantially parallel to the first central axis, and/or the at least one first detection component and the magnetic element are disposed in a straight line substantially parallel to the first central axis.

In an embodiment, the input device further includes at least one second detection component disposed on the mounting base and configured to detect a magnetic field intensity of the magnetic element.

In an embodiment, two second detection components are provided, and the two second detection components are spaced apart from the magnetic element in a direction substantially parallel to the first central axis, and/or one first detection component is provided, the first detection component and the two second detection components are disposed on a plane substantially perpendicular to the first central axis, and the two second detection components are disposed on two sides of the first detection component.

In an embodiment, one or both of the mounting base and the rotation support defines a guide hole, the guide hole extends in a direction substantially parallel to the first central axis, and the connection member is configured to movably pass through the guide hole.

In an embodiment, the guide hole includes a first guide hole defined by the mounting base and a second guide hole defined by the rotation support, the first guide hole is coaxial with the second guide hole.

According to various embodiments of the present disclosure, an input device is provided, including a mounting base, a rotation support, a grip mechanism, a connection member, a magnetic element, and at least one second detection component. The rotation support is rotatable relative to the mounting base around a first central axis. The grip mechanism is disposed on the rotation support and is transitionable between an open position and a close position. The connection member is connected with the grip mechanism and is movable in a direction substantially parallel to the first central axis with the opening and closing of the grip mechanism. The magnetic element is disposed on the connection member. The at least one second detection component is disposed on the mounting base and is configured to detect a magnetic field intensity of the magnetic element.

According to the input device provided by the embodiments of the present disclosure, the magnetic element is disposed on the connection member, and the connection member can move with the opening and closing action of the grip mechanism. Therefore, the position of the magnetic element can change with the opening and closing action of the grip mechanism, thereby changing the position relationship between the second detection component and the magnetic element, resulting in that the second detection component can detect changes in magnetic field intensity, and can obtain the included angle between members of the grip mechanism through data calculation. Therefore, the included angle between members of the grip mechanism can be measured without using other sensors for angle calibration.

In an embodiment, two second detection components are provided, and are spaced apart from the magnetic element in a direction substantially parallel to the first central axis.

According to various embodiments of the present disclosure, a surgical robot is provided, including an input device according to any one of the embodiments described above.

### BRIEF DESCRIPTION OF THE DRAWINGS

The following drawings of this application are incorporated herein as a part of this application to understand this application. Embodiments of the present disclosure and their descriptions are shown in the drawings to explain the principles of the present disclosure.

In the drawings:

FIG. 1 is a schematic perspective view of an input device of a surgical robot according to some embodiments of the present disclosure, where a first grip member and a second grip member are opened to a maximum included angle;

FIG. 2 is an exploded perspective view of the input device in FIG. 1;

FIG. 3 is another exploded perspective view of the input device in FIG. 1;

FIG. 4 is a schematic sectional view of the input device in FIG. 1, where the first grip member and the second grip member are opened to the maximum included angle;

FIG. 5 is another schematic sectional view of the input device in FIG. 1, where the first grip member and the second grip member are closed to a minimum included angle; and

FIG. 6 is a schematic diagram of a partial structure of a mounting base of the input device in FIG. 2, showing an induction board, a first detection component, and second detection components.

### DETAILED DESCRIPTION OF THE EMBODIMENTS

In the following description, a large number of specific details are set forth to provide a more thorough understanding of this application. However, it is apparent to those skilled in the art that the embodiments of the present disclosure can be implemented without the need for one or more of these details. In other examples, in order to avoid confusion with the embodiments of the present disclosure, some well-known technical features in the art have not been described.

In order to thoroughly understand the embodiments of the present disclosure, a detailed structure is set forth in the follow description. Obviously, the implementation of the embodiment of the present disclosure is not limited to the specific details familiar to those skilled in the art. It should be noted that the ordinal numbers such as “first” and “second” quoted in this application are only signs, and do not have any other meaning, such as a specific order. Moreover, for example, the term “first component” itself does not imply the existence of “second component”, and the term “second component” itself does not imply the existence of “first component”. The terms “upper”, “lower”, “front”, “rear”, “left” and “right” and similar expressions used in this application are for illustration purposes only, and are not for limitation.

An input device 100 and a surgical robot are provided according to some embodiments of the present disclosure. The surgical robot includes an input device 100, an end effector may be controlled by a doctor operating the input device 100 via remote communication and a computer, and the end effector may be a pair of forceps, a pair of scissors, a clamp, etc. Other structures of the surgical robot may be designed according to the existing technology in the field, and are not described in detail herein.

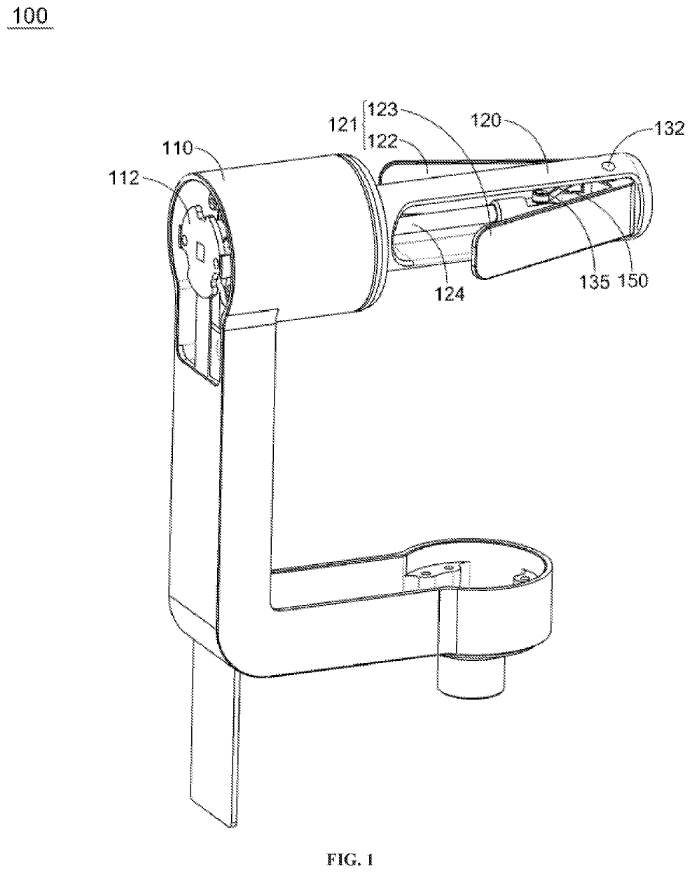

The input device 100 according to some embodiments of the present disclosure is described in detail with reference to FIG. 1 to FIG. 6.

As shown in FIG. 1 to FIG. 6, the input device 100 mainly includes a mounting base 110, a rotation support 120, a grip mechanism 121, a connection member 124, a magnetic element 140, at least one first detection component 114, at least one second detection component 115, an induction board 112, and a signal processing board 113.

The mounting base 110 is configured to fix the input device 100 on a desktop or other structure, and to mount a driving motor (not shown). The rotation support 120 is disposed on the mounting base 110 and rotatable around a first central axis relative to the mounting base 110. For example, the first central axis may be a central axis of the rotation support 120.

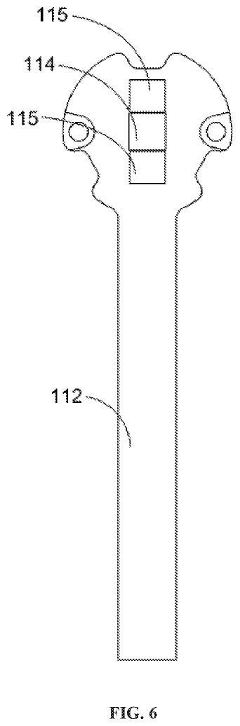

The grip mechanism 121 is disposed on the rotation support 120, and is capable of being elastically opened and closed relative to the rotation support 120. The connection member 124 is connected with the grip mechanism 121 and movable in a direction substantially parallel to the first central axis with the opening and closing of the grip mechanism 121. As shown in FIG. 6, the induction board 112 and the signal processing board 113 are mounted in the mounting base 110. The first detection component 114 and the second detection component 115 are mounted on the induction board 112. The signal processing board 113 is configured to receive and process information data transmitted by the induction board 112 (for example, the data detected by the first detection component 114 and the second detection component 115).

The first detection component 114 is configured to detect a magnetic field direction of the magnetic element. For example, the rotation angle of the rotation support 12 may be obtained based on a change of the rotation of the magnetic field with the magnetic element 140 detected by the first detecting component 114. The second detection component 115 is configured to detect the magnetic field intensity of the magnetic element 140. For example, the included angle between members of the grip mechanism 121 may be obtained based on a change in distance from the magnetic element 140 detected by the second detection component 115. Specifically, the magnetic element 140 may be a magnet. In some embodiments, the magnetic element may also be an alloy with magnetism. In some embodiments, the first detection component 114 and the magnetic element 140 are disposed in a straight line substantially parallel to the first central axis.

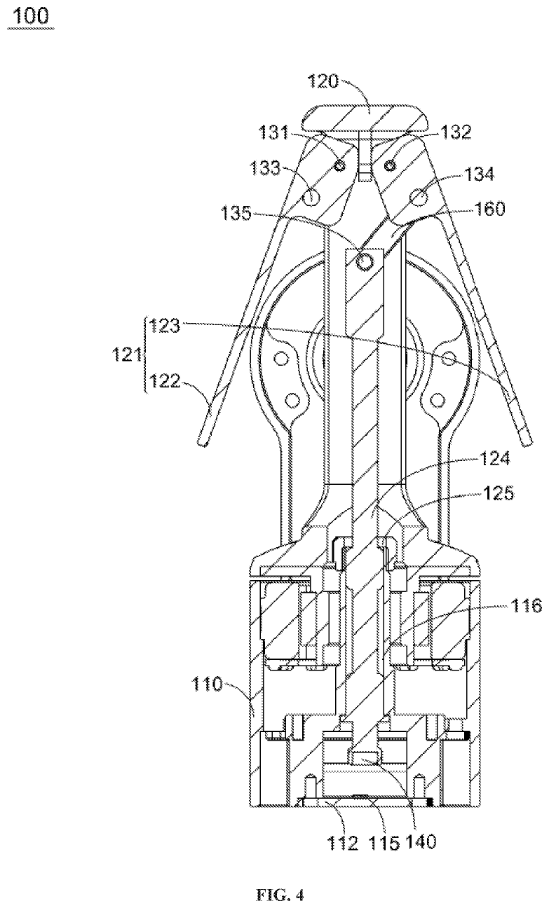

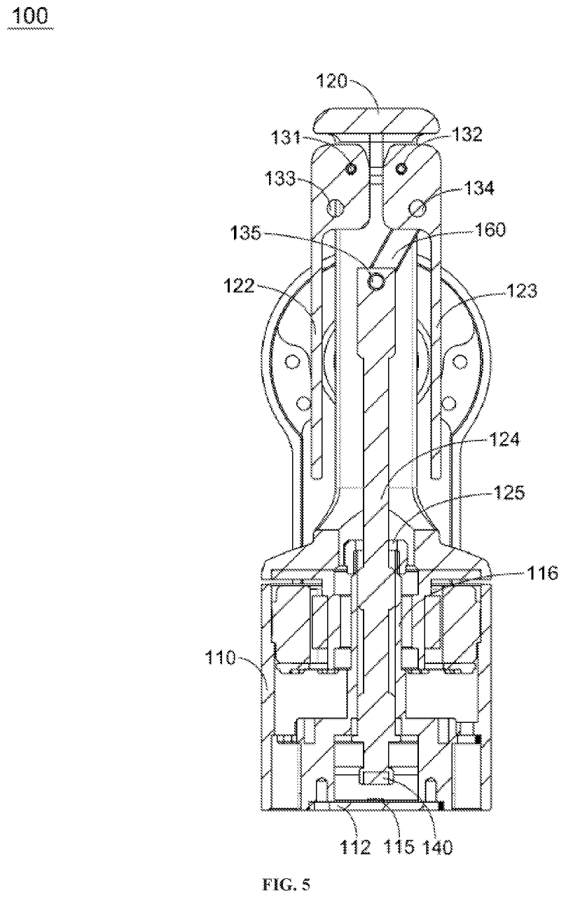

In this embodiment, as shown in FIG. 4 and FIG. 5, the magnetic element 140 is disposed on the connection member 124. As the grip mechanism 121 is being opened or closed, the connection member 124 moves together with the connection member 124 in a direction substantially parallel to the first central axis. In this way, the distance between the magnetic element 140 and the second detection component 115 is changed, and the included angle between members of the grip mechanism 121 is obtained by detection of the change.

Furthermore, the magnetic element 140 may also rotate with the rotation support 120 to change the relative rotation angle between the magnetic element 140 and the first detection component 114, so that the first detection component 114 may obtain the rotation angle of the grip mechanism 121 by detection of a rotation angle of the magnetic field of the magnetic element 140. In this embodiment, the included angle and the rotation angle of the grip mechanism 121 can be detected using only one magnetic element 140, thus reducing the number of detection components (such as sensor components). At the same time, the first detection component 114 and the second detection component 115 are disposed on the same signal processing board 113, which reduces the number of line connections, and the two detection components can be placed in the same coding system for processing.

As shown in FIG. 1 to FIG. 5, the grip mechanism 121 includes a first grip member 122 and a second grip member 123, the first grip member 122 and the second grip member 123 are pivotally connected to the rotation support 120, and the first grip member 122 and the second grip member 123 are cooperatively movable through a pair of spur gears, so that the first grip member 122 and the second grip member 123 pivot synchronously. One end of the first grip member 122 is pivotally disposed at one side of the rotation support 120. One end of the second grip member 123 is pivotally disposed on another side of the rotation support 120 opposite to the first grip member 122. The first grip member 122 and the second grip member 123 may transition (e.g., rotate) within the range of maximum included angle and minimum included angle. When the two grip members move closest to the rotation support 122, that is, the grip mechanism 121 is at a close position, the included angle between the two grip members is minimum, and when the two grip members move farthest from the rotation support 122, that is, the grip mechanism 121 is at an open position, the included angle between the two grip members is maximum. In this embodiment, the first grip member 122 and the second grip member 123 are symmetrically disposed about the first central axis. Those skilled in the art should understand that the installation positions of the first grip member 122 and the second grip member 123 are not limited to this embodiment.

In some embodiments, the magnetic element may be directly disposed on the rotation support or indirectly disposed on the rotation support through an intermediate connection member (not shown), so that the magnetic element can rotate with the rotation of the rotation support, the rotation angle of the magnetic element can be changed and detected by the first detection component to obtain the rotation angle of the rotation support.

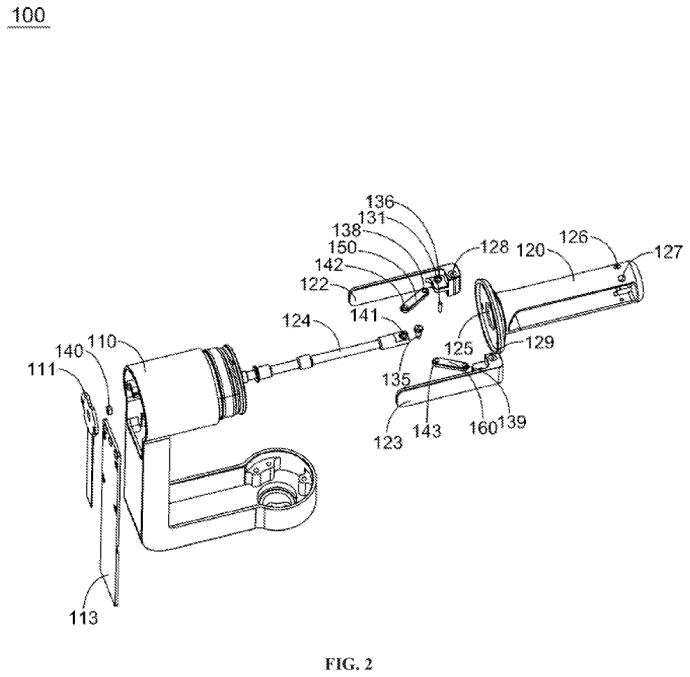

In this embodiment, one end of the connection member 124 is pivotally connected with the first grip member 122 and the second grip member 123, and the connection member 124 is rotatable around its central axis. As shown in FIG. 2 to FIG. 4, the magnetic element 140 is disposed at one end of the connection member 124 opposite to the end to which the first grip member 122 and the second grip member 123 are connected. The magnetic element 140 is spaced apart from the first detection component 114 and the second detection component 115 by a distance in a direction substantially parallel to the first central axis, so as to prevent the magnetic element 140 from interfering with the first detection component 114 and the second detection component 115 when the connection member 124 moves in a direction substantially parallel to the first central axis, and to avoid the magnetic element 140 being too close to the first detection component 114 and the second detection component 115, which may affect the detection of the first detection component 114 and the second detection component 115 (for example, affecting the detection accuracy).

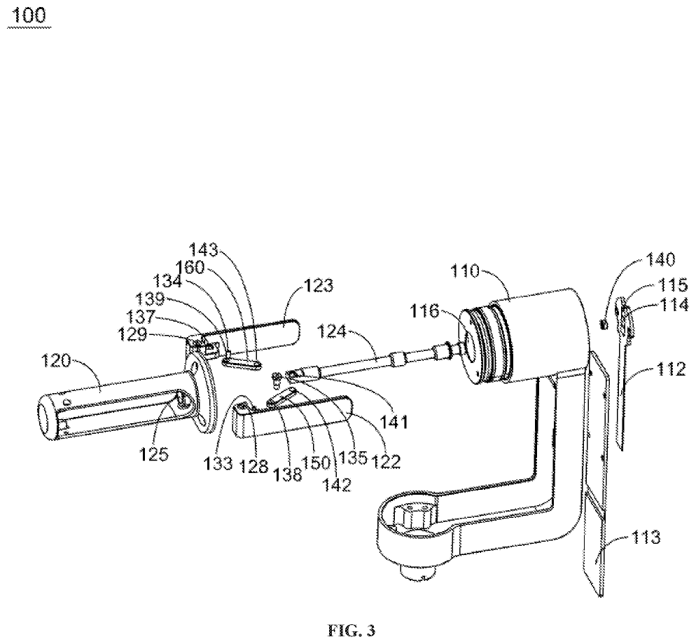

As shown in FIG. 3 and FIG. 6, in this embodiment, the input device 100 includes a first detection component 114 and two second detection components 115. In an embodiment, in the direction along the first central axis, the position of the first detection component 114 and the position of the magnetic element 140 are aligned. The first detection component 114 and the two second detection components 115 are disposed on a plane substantially perpendicular to the first central axis. The two second detection components 115 are disposed in a vertical array with the first detection component 114 located therebetween. The first detection component 114 and the second detection component 115 may be Hall sensing chips. It should be understood that the position relationship between the first detection component 114 and the second detection component 115 is not limited to this embodiment. As needed, the two second detection components 115 may be disposed in a horizontal array with the first detection component 114 located therebetween, or may be disposed at any other suitable position.

During detection, the magnetic field intensity detected by the second detection component 115 does not change in a linear relation with the change of the distance between the second detection component and the magnetic element, but the relation of the changes may be approximately regarded as linear within a certain range. In this embodiment, the detected magnetic field intensity within this range is taken as an effective basis to calculate or match the included angles between the first grip member 122 and the second grip member 123 at this magnetic field intensity. In addition, the first detection component 114 and the second detection component 115 are combined in a set of encoder system, which can simplify the detection scheme, simplify the structure of the input device 100, reduce the difficulty of wiring, and increase the reliability of the entire input device 100.

Guide holes are defined on the mounting base 110 and/or the rotation support 120, and extend in a direction substantially parallel to the first central axis. The connection member 124 may movably pass through the guide hole, for example, the connection member 124 may move along the extension direction of the guide hole. The connection member 124 may be configured as a shaft, a rod, an elongated shape or any suitable shape. As shown in FIG. 2 to FIG. 5, the guide holes include a first guide hole 116 defined by the mounting base 110 and a second guide hole 125 defined by the rotation support 120. The first guide hole 116 and the second guide hole 125 are both constructed as circular through holes, and the first guide hole 116 is coaxial with the second guide hole 125. In an embodiment, the central axes of the first guide hole 116 and the second guide hole 125 coincide with the first central axis.

The input device 100 further includes an elastic element (not shown), and opposite ends of the elastic element are connected to the first grip member 122 and the second grip member 123, respectively. The elastic element applies pushing forces to the first grip member 122 and the second grip member 123 that are opened or tended to be opened with the pushing force, so that the first grip member 122 and the second grip member 123 can be kept at the maximum opening position, that is, the maximum included angle, without external force. The elastic element may be a compression spring. In some embodiments, the elastic element may be elastic sheet or other elastically deformable connection members.

As shown in FIG. 1 to FIG. 5, the input device 100 further includes a first pin 131 and a second pin 132 substantially parallel to the first pin 131, and the first pin 131 is substantially perpendicular to the first central axis. One end of the first grip member 122 is pivotally connected to the rotation support 120 through the first pin 131, one end of the second grip member 123 is rotatably connected to the rotating shaft 120 through the second pin 132.

Specifically, a first mounting hole 126 and a second mounting hole 127 are defined by the rotating support 120, a first connecting hole 128 is correspondingly defined by the first grip member 122, and a second connecting hole 129 is correspondingly defined by the second grip member 123. Each of the first mounting hole 126, the second mounting hole 127, the first connecting hole 128 and the second connecting hole 129 may be configured as a circular through hole. The first pin 131 is inserted into the first mounting hole 126 and the first connecting hole 128 to pivotally connect the first grip member 122 to the rotation support 120. The second pin 132 is inserted into the second mounting hole 127 and the second connecting hole 129 to pivotally connect the second grip member 123 to the rotation support 120.

As shown in FIG. 3 to FIG. 5, the input device 100 further includes a first connecting rod 150, a second connecting rod 160, a third pin 133, a fourth pin 134 and a fifth pin 135. The third pin 133, the fourth pin 134 and the fifth pin 135 are substantially parallel to each other and substantially perpendicular to the first central axis. One end of the first connecting rod 150 is pivotally connected to the first grip member 122 through the third pin 133, and one end of the second connecting rod 160 is pivotally connected to the second grip member 123 through the fourth pin 134, the other end of the first connecting rod 150 and the other end of the second connecting rod 160 are pivotally connected to one end of the connection member 124 near the first grip member 122 and the second grip member 123 through the fifth pin 135.

Specifically, a third mounting hole 136 is defined by the first grip member 122, and a third connecting hole 138 is defined at one end of the first connecting rod 150. The third pin 133 is inserted into the third mounting hole 136 and the third connecting hole 138 to pivotally connect the first connecting rod 150 to the first grip member 122. A fourth mounting hole 137 is defined by the second grip member 123, and a fourth connecting hole 139 is defined at one end of the second connecting rod 160. The fourth pin 134 is inserted into the fourth mounting hole 137 and the fourth connecting hole 139 to pivotally connect the second connecting rod 160 to the second grip member 123. Each of the third mounting hole 136, the third connecting hole 138, the fourth mounting hole 137, and the fourth connecting hole 139 may be configured as a circular through hole.

A fifth mounting hole 141 is defined at one end of the connection member 124, a fifth connecting hole 142 is correspondingly defined at the other end of the first connecting rod 150, and a sixth connecting hole 143 is correspondingly defined at the other end of the second connecting rod 160. The fifth pin 135 is inserted into the fifth mounting hole 141, the fifth connecting hole 142 and the sixth connecting hole 143 to pivotally connect the other end of the first connecting rod 150 (i.e., the end farther away from the first grip member 122) and the other end of the second connecting rod 160 (i.e., the end farther away from the second grip member 123) to the connection member 124. Each of the fifth mounting hole 141, the fifth connecting hole 142 and the sixth connecting hole 143 may be configured as a circular through hole.

The working principle of the input device 100 according to the embodiments of the present disclosure is described in detail below.

As shown in FIG. 4, when the first grip member 122 and the second grip member 123 of the input device 100 are opened to the maximum included angle, that is, the grip mechanism 121 is at the open position, the magnetic element 140 is farthest from the first detection component 114 and the second detection component 115. When forces are exerted (by, e.g., fingers) on the first grip member 122 and the second grip member 123 so that the two grip members transition toward the minimum included angle, the first grip member 122 and the second grip member 123 exert forces on the first connecting rod 150 and the second connecting rod 160, respectively. In this way, the forces are transmitted to the connection member 124, which drive the connection member 124 to move linearly along its axial direction. As shown in FIG. 5, when the first grip member 122 and the second grip member 123 are closed to the minimum included angle, that is, the grip mechanism 121 is at the close position, the magnetic element 140 is closest to the first detection component 114 and the second detection component 115. During pivot of the first grip member 122 and the second grip member 123, the second detection component 115 can detect the magnetic field intensity of the magnetic element 140 at different positions, the data information detected by the second detection component 115 is transmitted to the signal processing board 113, and then processed by the signal processing board 113 to obtain the information about the included angle of the first grip member 122 and the second grip member 123.

When the rotation support 120 rotates, the first grip member 122, the second grip member 123, the connection member 124 and the magnetic element 140 rotate synchronously with the rotation support 120, and there are components of the magnetic field in both x and y directions in the plane where the first detection component 114 is located. The data information detected by the first detection component 114 is transmitted to the signal processing board 113 which calculates the current angle of the magnetic element 140 relative to the induction board 112 through data calculation. This angle reflects the current rotation angle of the rotation support 120. During the rotation of the rotation support 120, the included angles between the first grip member 122 and the second grip member 123 may change to move the connection member 124 and thus the magnetic element 140, resulting in a change in the position or distance of the magnetic element 140 relative to the first detection component 114. However, for each specific rotation angle of the rotation support 120, the calculation results of the components of the magnetic field in the x and y directions located in the plane where the first detection component 114 is located (for example, the directions of the components of the magnetic field) do not change with the change of the distance between the first detection component 114 and the magnetic element 140. Therefore, regardless of the included angles between the first grip member 122 and the second grip member 123, the rotation angle of the rotation support 120 relative to the mounting base 110 can be calculated and detected by the first detection component 114 and the signal processing board 113.

Unless otherwise defined, technical and scientific terms used herein have the same meaning as commonly understood by those skilled in the technical field of this application. The terminology used herein is only for describing specific implementation purposes and is not intended to limit the application. The terms such as “dispose” appearing in this specification can refer to either a component directly attached to another component or a component attached to another component through middleware. A feature described in one embodiment herein can be applied to another embodiment alone or in combination with other features, unless the feature is not applicable in the other embodiment or otherwise specified.

The application has been described by the above embodiments, but it should be understood that the above embodiments are only used for the purpose of illustration and description, and are not intended to limit the application to the described embodiments. It should be understood by those skilled in the art that many more variations and modifications can be made according to the teaching of this application, and these variations and modifications are all within the scope of protection claimed in this application.

What is claimed is:

1. An input device, comprising:
a mounting base;
a rotation support, rotatable relative to the mounting base around a first central axis;
a grip mechanism, disposed on the rotation support and transitionable between an open position and a close position;
a connection member, connected with the grip mechanism and movable in a direction substantially parallel to the first central axis with opening and closing of the grip mechanism;
a magnetic element, disposed on the rotation support or the connection member; and
at least one first detection component, disposed on the mounting base and configured to detect a magnetic field direction of the magnetic element.

2. The input device according to claim 1, wherein the grip mechanism comprises a first grip member and a second grip member, which are respectively pivotally disposed on the rotation support.

3. The input device according to claim 2, wherein the first grip member and the second grip member are disposed on two opposite sides of the rotation support respectively, and the first grip member and the second grip member are cooperatively movable, and/or
the connection member has one end to which the first grip member and the second grip member are pivotally connected, and one other end on which the magnetic element is disposed and which is located away from the first grip member and the second grip member.

4. The input device according to claim 2, further comprising a first pin and a second pin substantially parallel to the first pin, wherein the first grip member has one end pivotally connected to the rotation support through the first pin, and the second grip member has one end pivotally connected to the rotation support through the second pin.

5. The input device according to claim 4, further comprising a first connecting rod and a second connecting rod, wherein the first connecting rod has two ends pivotally connected to the first grip member and the connection member, respectively, and the second connecting rod has two ends pivotally connected to the second grip member and the connection member, respectively.

6. The input device according to claim 5, further comprising a third pin, a fourth pin and a fifth pin, which are substantially parallel to each other, wherein one of the two ends of the first connecting rod is pivotally connected to the first grip member through the third pin, one of the two ends of the second connecting rod is pivotally connected to the second grip member through the fourth pin, and the other one of the two ends of the first connecting rod and the other one of the two ends of the second connecting rod are pivotally connected to the connection member through the fifth pin.

7. The input device according to claim 6, wherein
the third pin is substantially parallel to the first pin, and/or
a central axis of the third pin is substantially perpendicular to the first central axis.

8. The input device according to claim 1, wherein
each of the at least one first detection component is spaced apart from the magnetic element in a direction substantially parallel to the first central axis, and/or
the at least one first detection component and the magnetic element are disposed in a straight line substantially parallel to the first central axis.

9. The input device according to claim 1, further comprising at least one second detection component disposed on the mounting base and configured to detect a magnetic field intensity of the magnetic element.

10. The input device according to claim 9, wherein two second detection components are provided,
wherein the two second detection components are spaced apart from the magnetic element in a direction substantially parallel to the first central axis, and/or
wherein one first detection component is provided, the first detection component and the two second detection components are disposed on a plane substantially perpendicular to the first central axis, and the two second detection components are disposed on two sides of the first detection component.

11. The input device according to claim 1, wherein one or both of the mounting base and the rotation support defines a guide hole, the guide hole extends in a direction substantially parallel to the first central axis, and the connection member is configured to movably pass through the guide hole.

12. The input device according to claim 11, wherein the guide hole comprises a first guide hole defined by the mounting base and a second guide hole defined by the rotation support, and the first guide hole is coaxial with the second guide hole.

13. An input device, comprising:
a mounting base;
a rotation support, rotatable relative to the mounting base around a first central axis;
a grip mechanism, disposed on the rotation support and transitionable between an open position and a close position;
a connection member, connected with the grip mechanism and movable in a direction substantially parallel to the first central axis with opening and closing of the grip mechanism;
a magnetic element, disposed on the connection member; and
at least one second detection component, disposed on the mounting base, and configured to detect a magnetic field intensity of the magnetic element.

14. The input device according to claim 13, wherein two second detection components are provided, and the two second detection components are spaced apart from the magnetic element in a direction substantially parallel to the first central axis.

15. The input device according to claim 13, wherein the grip mechanism comprises a first grip member and a second grip member, which are respectively pivotally disposed on the rotation support.

16. The input device according to claim 15, wherein the first grip member and the second grip member are disposed on two opposite sides of the rotation support respectively, and the first grip member and the second grip member are cooperatively movable, and/or
the connection member has one end to which the first grip member and the second grip member are pivotally connected, and one other end on which the magnetic element is disposed and which is located away from the first grip member and the second grip member.

17. The input device according to claim 15, further comprising a first pin and a second pin substantially parallel to the first pin, wherein the first grip member has one end pivotally connected to the rotation support through the first pin, and the second grip member has one end pivotally connected to the rotation support through the second pin.

18. The input device according to claim 17, further comprising a first connecting rod and a second connecting rod, wherein the first connecting rod has two ends pivotally connected to the first grip member and the connection member, respectively, and the second connecting rod has two ends pivotally connected to the second grip member and the connection member, respectively.

19. A surgical robot, comprising an input device according to claim 1.

20. A surgical robot, comprising an input device according to claim 13.
